(19) United States
(12) Patent Application Publication
(10) Pub. No.: US 2023/0167071 A1
(43) Pub. Date: Jun. 1, 2023

(54) COMPOUNDS WITH ANTIMALARIAL ACTIVITY

(71) Applicant: UNIVERSITY OF PUERTO RICO, San Juan, PR (US)

(72) Inventors: Adelfa E. Serrano Brizuela, San Juan, PR (US); Emilee E. Colón Lorenzo, San Juan, PR (US); Jürgen Bosch, Cleveland Heights, OH (US)

(21) Appl. No.: 17/603,217

(22) PCT Filed: Feb. 10, 2020
(86) PCT No.: PCT/US2020/017505

§ 371 Date: Oct. 12, 2021

Related U.S. Application Data
(60) Provisional application No. 62/833,353, filed on Apr. 12, 2019.

(51) Int. Cl. C07D 239/26; C07D 215/14; C07D 487/04; A61P 33/06; C07C 205/06

(52) U.S. Cl. CPC C07D 239/26; C07D 215/14; C07D 487/04; A61P 33/06; C07C 205/06

(57) ABSTRACT

Methods of inhibiting growth of a Plasmodium species, treating malaria, and inhibiting a glutathione S-transferase are provided. The methods include inhibiting growth of a Plasmodium species comprising contacting a Plasmodium species with a compound as disclosed herein. The methods also include treating malaria comprising administering a compound as disclosed herein to a human or animal patient, preferably a human patient, in need thereof. The methods further include inhibiting a glutathione S-transferase (GST) comprising contacting a GST with a compound as disclosed herein. Also provided are compounds for use in the disclosed methods.

### CROSS-REFERENCE TO RELATED APPLICATIONS

This application is the National Stage filing under 35 U.S.C. 371 of International Application No. PCT/US2020/017505, filed Feb. 10, 2020, which claims the benefit of priority to U.S. Provisional Patent Application No. 62/833,353, filed Apr. 12, 2019, the entire content of which is hereby incorporated by reference in its entirety.

### STATEMENT REGARDING FEDERALLY SPONSORED RESEARCH OR DEVELOPMENT

This invention was made with government support under contract numbers G12MD007600 and U54MD007600 awarded by the National Institutes of Health-National Institute on Minority Health and Health Disparities (NIH-NIMHD) and under contract number GM08224 awarded by the National Institutes of Health-National Institute of General Medical Sciences (NIH-NIGMS). The government has certain rights in the invention.

### BACKGROUND

Malaria is a global health problem with an estimated 219 million cases and nearly half a million deaths in 2017. The resistance of Plasmodium parasites to almost all available antimalarial drugs represents a real problem and jeopardizes malaria elimination efforts. Consequently, there is an urgent need to identify new and effective drugs against validated targets as well as to identify and validate novel antimalarial targets. The discovery and design of new chemical scaffolds, especially those against validated biological targets are urgently needed.

Plasmodium falciparum glutathione S-transferase (PfGST) is an attractive antimalarial drug target recently validated as an essential gene, critical for parasite survival. The GST protein has multiple biological roles, including cellular detoxification against toxic molecules, and cell protection against oxidative stress. Additionally, GTS conjugates glutathione (GSH) to a wide range of hydrophobic and electrophilic molecules making them less harmful to active transport out of the cell. Plasmodium GST inhibitors potentiate the accumulation of chloroquine metabolites leading to parasite death. Additionally, ellagic acid, a specific GST inhibitor, has been shown to inhibit P. falciparum and P. vinckei petteri parasite growth.

The three-dimensional structure of PfGST has been solved and is reported to be a dimeric enzyme with two binding sites, the G-site which binds GSH, and the H-site that binds a variety of substrates. Phylogenetic and structural analyses classified the PfGST as a sigma class GST and bioinformatics analysis showed that Plasmodium spp. GSTs are highly conserved. The main structural difference between PfGST and human GST are at the H-site which is more exposed and has an atypical extra loop connecting the α-4 and α-5 helices which are involved in dimer formation.

### SUMMARY

The present application describes methods of inhibiting growth of a Plasmodium species, methods of treating malaria, and methods of inhibiting a glutathione S-transferase using compounds as disclosed herein. Also described are compounds for use in the disclosed methods.

According to one non-limiting aspect of the present disclosure, an example embodiment is a method of inhibiting growth of a Plasmodium species comprising contacting a Plasmodium species with a compound as disclosed herein.

According to one non-limiting aspect of the present disclosure, an example embodiment is a method for treating malaria comprising administering a compound as disclosed herein to a human or animal patient, preferably a human patient, in need thereof.

According to one non-limiting aspect of the present disclosure, an example embodiment is a method of inhibiting a glutathione S-transferase (GST) comprising contacting a GST with a compound as disclosed herein.

Additional features and advantages are described herein, and will be apparent from the following Detailed Description and the Figures.

### BRIEF DESCRIPTION OF THE DRAWINGS

Features and advantages of the compositions and methods described herein may be better understood by reference to the accompanying drawing in which:

FIG. 1A is a schematic drawing showing steps of the structure-based in silico screening process of the ChemBridge Hit2Lead library to identify Plasmodium berghei glutathione S-transferase (PbGST) inhibitors.

FIG. 1B and FIG. 1C are graphs showing screening of compounds at 10 μM in a P. berghei in vitro drug assay. Three compounds showed >50% of parasite growth inhibition as indicated by arrows. Data represents one biological experiment in triplicate each (bars=SD).

FIG. 2A shows the chemical structure of CB-6.

FIG. 2B is a graph showing a dose-response curve of compound CB-6. Data represents four independent experiments in triplicate each (bars=SEM).

FIG. 2C shows the chemical structure of CB-19.

FIG. 2D is a graph showing a dose-response curve of compound CB-19. Data represents four independent experiments in triplicate each (bars=SEM).

FIG. 3A shows the chemical structure of CB-27.

FIG. 3B is a graph showing a dose-response curve of compound CB-27. Data from P. berghei represent four independent experiments in triplicate (bars are SEM) and data from P. falciparum Dd2 represents six independent experiments in duplicate (bars are SEM).

FIG. 4A is a predicted binding mode of compound CB-27 in the PbGST H-site.

FIG. 4B is graph showing inhibition of GST activity by compound CB-27 in a crude P. berghei protein extract. The PbGST activity was evaluated using protein parasite extracts from blood stages. The effect of compound CB-27 was determined using four different concentrations (0, 1, 10, and 50 μM). The enzymatic activity was determined in four independent experiments (one replica), and bars represents SEM.

FIG. 4C is graph showing inhibition of GST activity by compound CB-27 in human placenta GST. The effect of compound CB-27 was determined using four different concentrations (0, 1, 10, and 50 μM).

FIG. 4D is graph showing inhibition of GST activity by S-hexylglutathione in a crude P. berghei protein extract. The PbGST activity was evaluated using protein parasite extracts from blood stages. The effect of S-hexylglutathione was determined using three different concentrations (5, 50, and 250 μM). The enzymatic activity was determined in two independent experiments (one replica), and bars represents SEM.

FIG. 4E is graph showing inhibition of GST activity by compound S-hexylglutathione in human placenta GST. The effect of S-hexylglutathione was determined using three different concentrations (5, 50, and 250 μM). The enzymatic activity was determined in three independent experiments (one replica), and bars represents SEM.

FIG. 5 shows compounds with similar shape and electrostatic properties to compound CB-27.

FIG. 6A is a graph showing screening of compounds at 10 μM in a P. berghei in vitro luminescence drug assay. Six compounds showed >50% of parasite growth inhibition as indicated by arrows. Data represents one experiment in triplicate each (bars=SD).

FIG. 6B, FIG. 6C, FIG. 6D, FIG. 6E, FIG. 6F, and FIG. 6G are dose-response curves of compounds CB-41, CB-50, CB-53, CB-58, CB-59, and CB-61, respectively. Data represents four independent experiments in triplicate each, bars represent SEM.

FIG. 7 is a graph showing lytic activity of compounds. The positive control for 100% cell lysis is saponin at 100 μg/mL; the negative control for no cell lysis is blood (1% hematocrit)+DPBS; the vehicle is DPBS as blank. Compounds were tested at serial dilutions from 0.2× to 100× folds of their EC₅₀ value from antimalarial dose-response curves. Data represents three independent experiments in triplicate each, bars represent SEM.

### DETAILED DESCRIPTION

Compounds and methods of inhibiting growth of a Plasmodium species, treating malaria, and inhibiting a glutathione S-transferase are disclosed herein.

The method of inhibiting growth of a Plasmodium species involves contacting a Plasmodium species with a compound as disclosed herein. In some cases, the Plasmodium species is Plasmodium falciparum, Plasmodium vivax, Plasmodium malariae, Plasmodium ovale, Plasmodium knowlesi, or Plasmodium berghei. In some cases, the Plasmodium species is a multidrug-resistant Plasmodium strain. In some cases, the method of inhibiting growth of a Plasmodium species further comprises co-contacting the Plasmodium species with a compound selected from the group consisting of chloroquine, atovaquone-proguanil, artemether-lumefantrine, mefloquine, quinine, quinidine, doxycycline, clindamycin, artesunate, and combinations thereof.

The method for treating malaria comprises administering a compound as disclosed herein to a human or animal patient, preferably a human patient, in need thereof. In some cases, the patient is infected with a Plasmodium species, such as Plasmodium falciparum, Plasmodium vivax, Plasmodium malariae, Plasmodium ovale, Plasmodium knowlesi, or Plasmodium berghei. In some cases, the Plasmodium species is a multidrug-resistant Plasmodium strain. In some cases, the method of treating malaria further comprises co-administering a compound selected from the group consisting of chloroquine, atovaquone-proguanil, artemether-lumefantrine, mefloquine, quinine, quinidine, doxycycline, clindamycin, artesunate, and combinations thereof.

The method for inhibiting a glutathione S-transferase (GST) comprises contacting a GST with a compound as disclosed herein. In some cases, the GST is from a Plasmodium species, such as Plasmodium falciparum, Plasmodium vivax, Plasmodium malariae, Plasmodium ovale, Plasmodium knowlesi, or Plasmodium berghei. In some cases, the Plasmodium species is a multidrug-resistant Plasmodium strain. In some cases, the method of inhibiting a GST further comprises co-contacting the GST with a compound selected from the group consisting of chloroquine, atovaquone-proguanil, artemether-lumefantrine, mefloquine, quinine, quinidine, doxycycline, clindamycin, artesunate, and combinations thereof.

As used herein, the term “aryl” refers to an all-carbon monocyclic or fused-ring polycyclic groups of 6 to 10 carbon atoms having a completely conjugated pi-electron system. Exemplary aryl groups include, but are not limited to, phenyl and naphthylenyl. Aryl groups may be unsubstituted or substituted with groups including, but not limited to, alkyl, cycloalkyl, heterocycloalkyl, aryl, heteroaryl, hydroxy, alkoxy, aryloxy, mercapto, alkylthio, arylthio, cyano, halo, carbonyl, nitro, and amino.

As used herein, the term “heteroaryl” refers to a monocyclic or fused ring group of 5 to 10 ring atoms containing one, two, three or four ring heteroatoms selected from nitrogen, oxygen and sulfur, the remaining ring atoms being carbon atoms, and also having a completely conjugated pi-electron system. Heteroaryl groups may be unsubstituted, or substituted as described for aryl. Exemplary heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazolyl, and the like.

The present disclosure provides compounds of formulae I, II, and III:

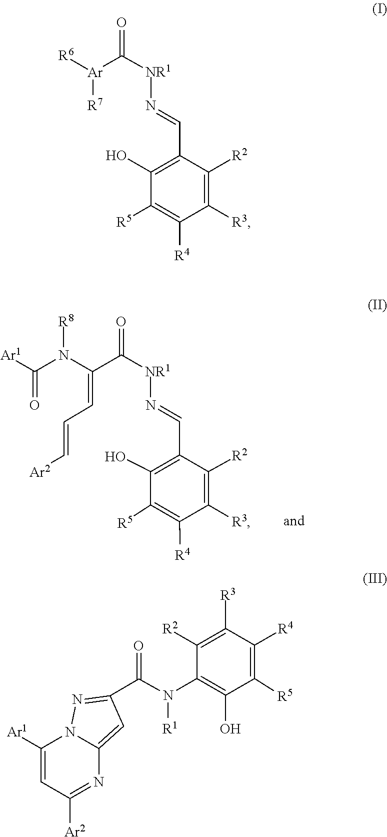

In formulae I, II, and III, R¹ is H or C₁₋₃ alkyl, such as methyl, ethyl, n-propyl, or isopropyl. In some cases, R², R³, R⁴, and R⁵ are independently H, C₁₋₃ alkyl, methyl, ethyl, n-propyl, isopropyl, C₁₋₃ alkoxy, methoxy, ethoxy, propoxy, n-propoxy, isopropoxy F, Cl, Br, or I. In some cases, R² and R³, R³ and R⁴, or R⁴ and R⁵, taken together with the carbon atoms to which they are attached, form a 5- or 6-membered aryl or heteroaryl ring. In some cases, the 5- or 6-membered aryl or heteroaryl ring is a benzene ring. In some cases, R² and R³, together with the carbon atoms to which they are attached, form a benzene ring, and R⁴ and R⁵ are H. In some cases, R², R³, and R⁴ are H, and R⁵ is methoxy. In some cases, R², R³, R⁴, and R⁵ are all H. In some cases, R³ is Br and R², R⁴, and R⁵ are H. In some cases, R³ is Cl and R², R⁴, and R⁵ are H.

In formula I, Ar is a 5- to 10-membered aryl or heteroaryl ring. In some cases, Ar is phenyl, pyridinyl, pyridazinyl, pyrimidinyl, pyrazinyl, triazinyl, pyrrolyl, imidazolyl, pyrazolyl, triazolyl, quinolinyl, or isoquinolinyl. In some cases, Ar is

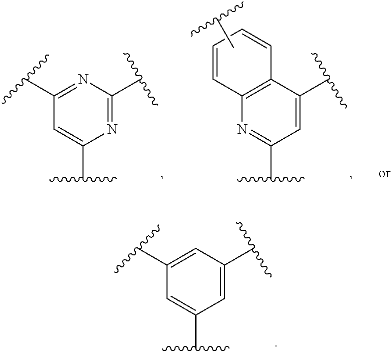

In formula I, R⁶ is H, NO₂, or a 5- or 6-membered aryl or heteroaryl ring, and R⁷ is NO₂ or a 5- or 6-membered aryl or heteroaryl ring. In some cases, the 5- or 6-membered aryl or heteroaryl ring is phenyl. In some cases, R⁶ and R⁷ are both phenyl. In some cases, R⁶ and R⁷ are both NO₂. In some cases R⁶ is H and R⁷ is phenyl.

In some cases, the compound of formula I is

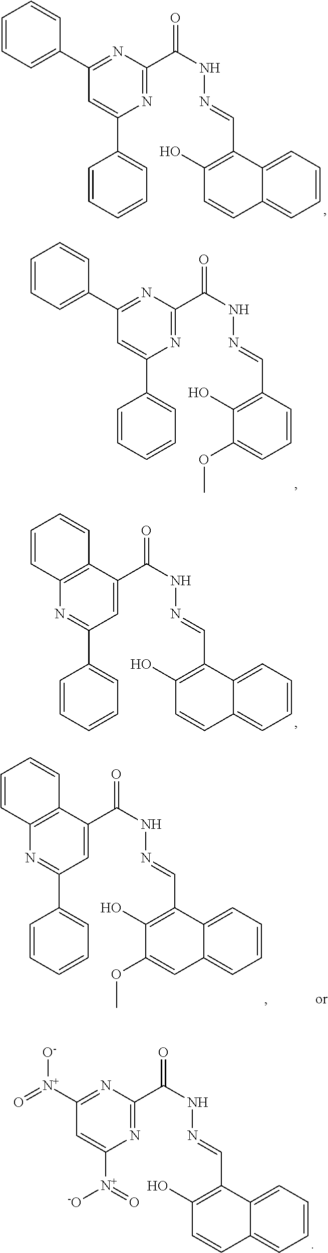

In formula II, R⁸ is H or C₁₋₃ alkyl, such as methyl, ethyl, n-propyl, or isopropyl.

In formulae II and III, Ar¹ and Ar² are independently NO₂ or a 5- or 6-membered aryl or heteroaryl ring. In some cases the 5- or 6-membered aryl or heteroaryl ring is phenyl. In some cases, Ar¹ and Ar² are both phenyl.

In some cases, the compound of formula II is

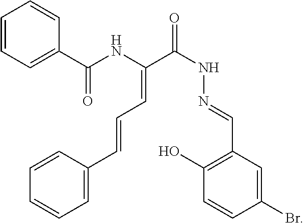 [SMILES: O=C(N/N=C/c1cc(Br)ccc1O)/C(=C/C=C/c1ccccc1)NC(=O)c1ccccc1]

In some cases, the compound of formula III is

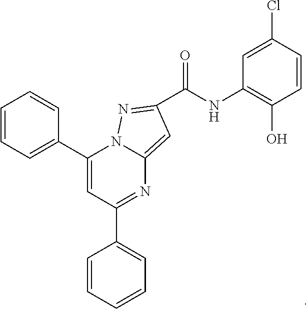 [SMILES: O=C(Nc1cc(Cl)ccc1O)c1cc2nc(-c3ccccc3)cc(-c3ccccc3)n2n1]

In an embodiment of the present disclosure, which may be combined with any other embodiment listed herein unless specified otherwise, a method of inhibiting growth of a Plasmodium species comprises contacting a Plasmodium species with a compound as disclosed herein. In an embodiment of the present disclosure, which may be combined with any other embodiment listed herein unless specified otherwise, the Plasmodium species is Plasmodium falciparum, Plasmodium vivax, Plasmodium malariae, Plasmodium ovale, Plasmodium knowlesi, or Plasmodium berghei. In an embodiment of the present disclosure, which may be combined with any other embodiment listed herein unless specified otherwise, the Plasmodium species is a multidrug-resistant Plasmodium strain. In an embodiment of the present disclosure, which may be combined with any other embodiment listed herein unless specified otherwise, the method of inhibiting growth of a Plasmodium species further comprises co-contacting the Plasmodium species with a compound selected from the group consisting of chloroquine, atovaquone-proguanil, artemether-lumefantrine, mefloquine, quinine, quinidine, doxycycline, clindamycin, artesunate, and combinations thereof.

In an embodiment of the present disclosure, which may be combined with any other embodiment listed herein unless specified otherwise, a method for treating malaria comprises administering a compound as disclosed herein to a human or animal patient, preferably a human patient, in need thereof. In an embodiment of the present disclosure, which may be combined with any other embodiment listed herein unless specified otherwise, the method of treating malaria further comprises co-administering a compound selected from the group consisting of chloroquine, atovaquone-proguanil, artemether-lumefantrine, mefloquine, quinine, quinidine, doxycycline, clindamycin, artesunate, and combinations thereof.

In an embodiment of the present disclosure, which may be combined with any other embodiment listed herein unless specified otherwise, a method for inhibiting a glutathione S-transferase (GST) comprises contacting a GST with a compound as disclosed herein. In an embodiment of the present disclosure, which may be combined with any other embodiment listed herein unless specified otherwise, the GST is from a Plasmodium species.

In an embodiment of the present disclosure, which may be combined with any other embodiment listed herein unless specified otherwise, a compound selected from the group consisting of formulae I, II, and III is provided:

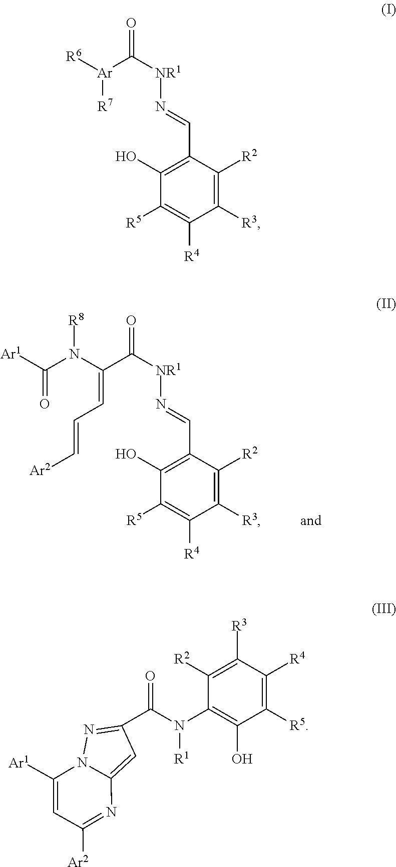

In an embodiment of the present disclosure, which may be combined with any other embodiment listed herein unless specified otherwise, R¹ is H or C₁₋₃ alkyl; R², R³, R⁴, and R⁵ are independently H, C₁₋₃ alkyl, C₁₋₃ alkoxy, F, Cl, Br, and I, or R² and R³, R³ and R⁴, or R⁴ and R⁵, taken together with the carbon atoms to which they are attached, form a 5- or 6-membered aryl or heteroaryl ring; Ar is a 5- to 10-membered aryl or heteroaryl ring; R⁶ is H, NO₂, or a 5- or 6-membered aryl or heteroaryl ring; R⁷ is NO₂ or a 5- or 6-membered aryl or heteroaryl ring; R⁸ is H or C₁₋₃ alkyl; and Ar¹ and Ar² are independently NO₂ or a 5- or 6-membered aryl or heteroaryl ring.

In an embodiment of the present disclosure, which may be combined with any other embodiment listed herein unless specified otherwise, R¹ is H.

In an embodiment of the present disclosure, which may be combined with any other embodiment listed herein unless specified otherwise, R² and R³, together with the carbon atoms to which they are attached, form a benzene ring.

In an embodiment of the present disclosure, which may be combined with any other embodiment listed herein unless specified otherwise, R⁴ and R⁵ are H.

In an embodiment of the present disclosure, which may be combined with any other embodiment listed herein unless specified otherwise, at least one of R², R³, R⁴, and R⁵ is methoxy.

In an embodiment of the present disclosure, which may be combined with any other embodiment listed herein unless specified otherwise, R², R³, R⁴, and R⁵ are H.

In an embodiment of the present disclosure, which may be combined with any other embodiment listed herein unless specified otherwise, R⁵ is methoxy and R², R³, and R⁴ are H.

In an embodiment of the present disclosure, which may be combined with any other embodiment listed herein unless specified otherwise, R², R³, R⁴, and R⁵ are H.

In an embodiment of the present disclosure, which may be combined with any other embodiment listed herein unless specified otherwise, Ar is phenyl, pyridinyl, pyridazinyl, pyrimidinyl, pyrazinyl, triazinyl, pyrrolyl, imidazolyl, pyrazolyl, triazolyl, quinolinyl, or isoquinolinyl.

In an embodiment of the present disclosure, which may be combined with any other embodiment listed herein unless specified otherwise, Ar is

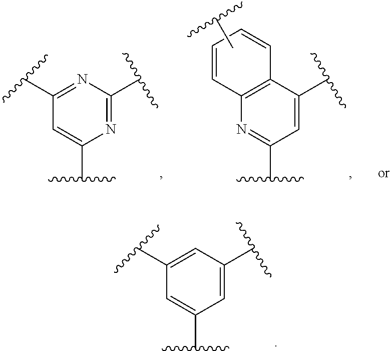

In an embodiment of the present disclosure, which may be combined with any other embodiment listed herein unless specified otherwise, R⁶ and R⁷ are both NO₂.

In an embodiment of the present disclosure, which may be combined with any other embodiment listed herein unless specified otherwise, R⁶ and R⁷ are both phenyl.

In an embodiment of the present disclosure, which may be combined with any other embodiment listed herein unless specified otherwise, R⁶ is H and R⁷ is phenyl.

In an embodiment of the present disclosure, which may be combined with any other embodiment listed herein unless specified otherwise, Ar¹ and Ar² are both phenyl.

In an embodiment of the present disclosure, which may be combined with any other embodiment listed herein unless specified otherwise, R⁸ is H.

In an embodiment of the present disclosure, which may be combined with any other embodiment listed herein unless specified otherwise, R², R³, R⁴, and R⁵ is Br and the others of one of R², R³, R⁴, and R⁵ are H.

In an embodiment of the present disclosure, which may be combined with any other embodiment listed herein unless specified otherwise, R³ is Br and R², R⁴, and R⁵ are H.

In an embodiment of the present disclosure, which may be combined with any other embodiment listed herein unless specified otherwise, one of R², R³, R⁴, and R⁵ is Cl and the others of one of R², R³, R⁴, and R⁵ are H.

In an embodiment of the present disclosure, which may be combined with any other embodiment listed herein unless specified otherwise, R³ is Cl and R², R⁴, and R⁵ are H.

In an embodiment of the present disclosure, which may be combined with any other embodiment listed herein unless specified otherwise, the compound is selected from the group consisting of:

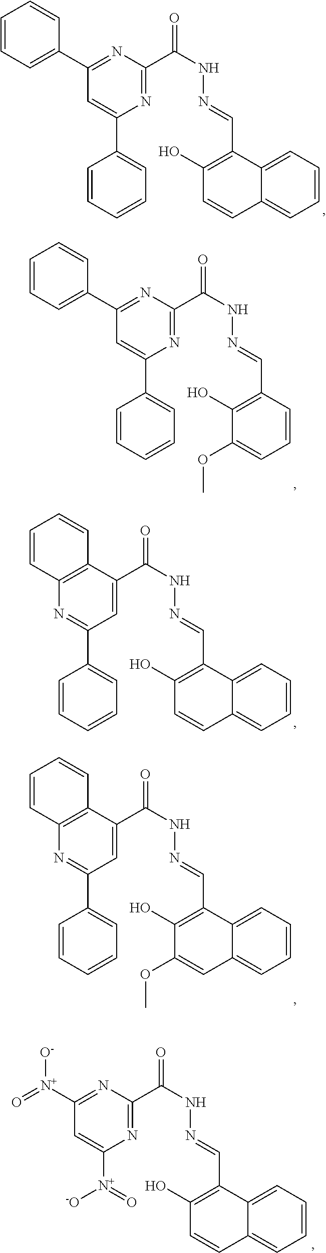

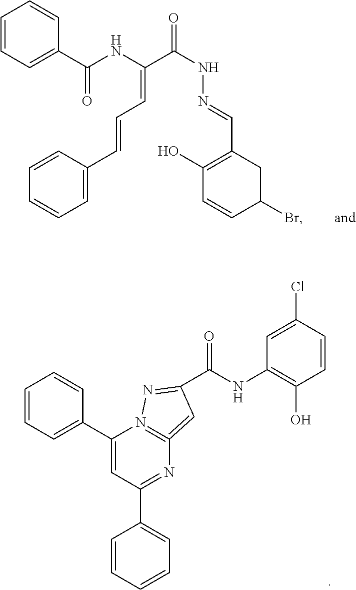

In an embodiment of the present disclosure, which may be combined with any other embodiment listed herein unless specified otherwise, a compound selected from the group consisting of formulae I, II, and III is provided:

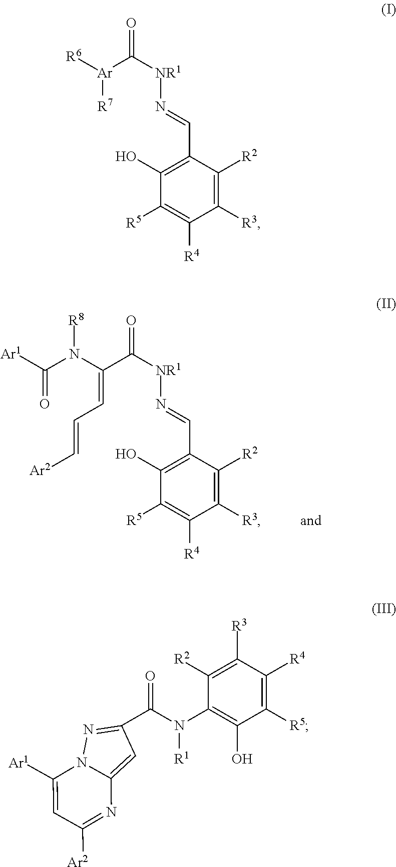

wherein R¹ is H or C₁₋₃ alkyl; R², R³, R⁴, and R⁵ are independently H, C₁₋₃ alkyl, C₁₋₃ alkoxy, F, Cl, Br, and I, or R² and R³, R³ and R⁴, or R⁴ and R⁵, taken together with the carbon atoms to which they are attached, form a 5- or 6-membered aryl or heteroaryl ring; Ar is a 5- to 10-membered aryl or heteroaryl ring; R⁶ is H, NO₂, or a 5- or 6-membered aryl or heteroaryl ring; R⁷ is NO₂ or a 5- or 6-membered aryl or heteroaryl ring; R⁸ is H or C₁₋₃ alkyl; and Ar¹ and Ar² are independently NO₂ or a 5- or 6-membered aryl or heteroaryl ring; with the proviso that the compound is not

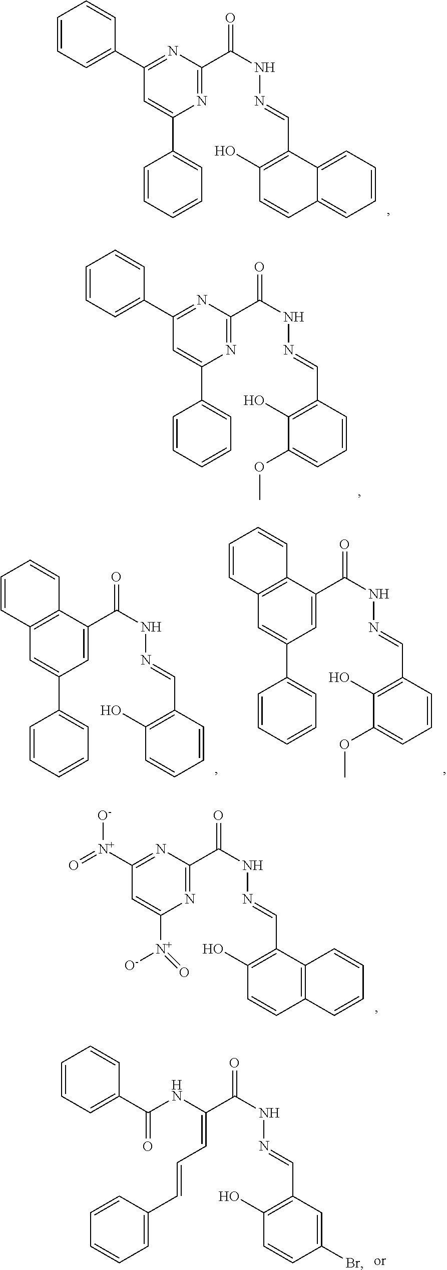

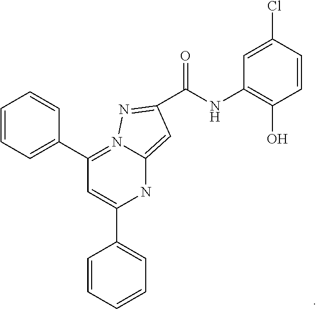

### Examples

### Example 1—Structure-Based in Silico Screening

Materials and Methods. Structure-based in silico screening was done using the PbGST homology model previously generated and the ChemBridge Hit2Lead library to identify potential inhibitors. The structure-based method was done using the PbGST model as the target protein and compounds from the Hit2Lead library from ChemBridge Corporation (http://www.hit2lead.com). Both the G and H binding sites of the PbGST protein were analyzed in the in silico screening. Docking analysis was done using the OpenEye Scientific software package (www.eyesopen.com) under standard parameters. The structure-based in silico screening FILTER tool was used to select compounds that comply with ADMET properties. A total of 2,000 conformers were generated for each compound using the OMEGA2 tool and docking analyses using the Fast Rigid Exhaustive Docking (FRED) with the standard parameters to examine all protein-ligand poses and filters for complementarity and chemical feature. Finally, molecular modeling and visualization using the VIDA tool were carried out and virtual hits were selected according to the formation of hydrogen bonds by ligand atoms with amino acid residues at the PbGST binding sites, selecting the best conformational and energetic favorable interactions.

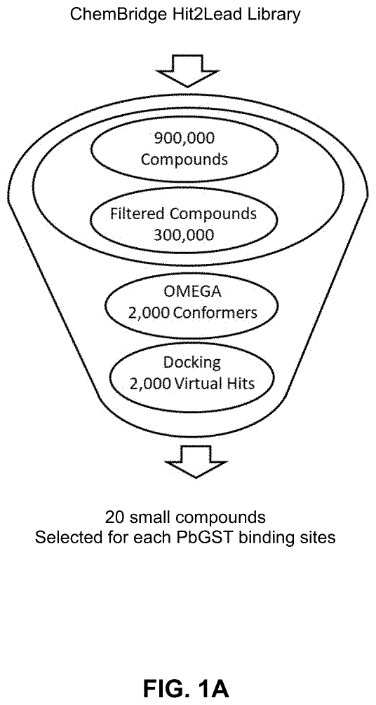

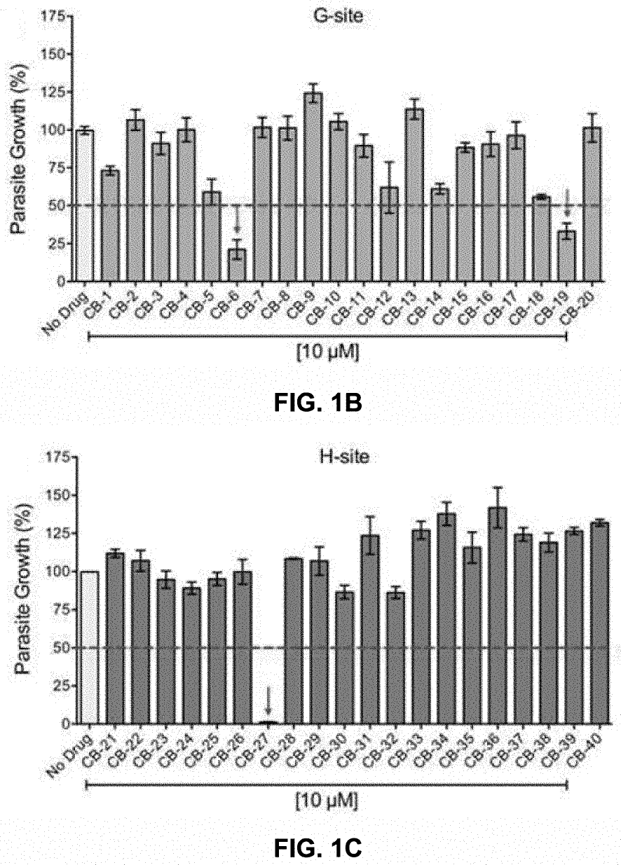

Results. A structure-based in silico screening was used against the structural model of the PbGST to identify virtual hits that potentially inhibited the selected drug target. Through a comprehensive pipeline structure-based in silico screening against the ChemBridge Hit2Lead library, a total of 2,000 virtual hits for each PbGST binding sites were identified (FIG. 1A). Twenty small compounds showing favorable binding interactions for each binding sites (40 compounds in total) were carefully chosen by docking score and molecular visualization. The molecular weight, predicted binding site, and docking score are provided in Table 1. Docking analyses were scored using the Chemgauss 4 scoring functions.

TABLE 1

| ID | g/mol | Binding Site | Docking score |
|---|---|---|---|
| CB-1 | 397.5 | G-site | −9.6979 |
| CB-2 | 438.6 | G-site | −9.1567 |
| CB-3 | 357.5 | G-site | −9.0084 |
| CB-4 | 412.5 | G-site | −9.7125 |
| CB-5 | 361.4 | G-site | −8.6675 |
| CB-6 | 499.6 | G-site | −8.5721 |
| CB-7 | 398.5 | G-site | −8.3073 |
| CB-8 | 398.5 | G-site | −8.1264 |
| CB-9 | 408.5 | G-site | −7.8427 |
| CB-10 | 355.4 | G-site | −8.3947 |
| CB-11 | 543.5 | G-site | −8.0503 |
| CB-12 | 452.5 | G-site | −7.1563 |
| CB-13 | 373.8 | G-site | −7.7669 |
| CB-14 | 418.9 | G-site | −7.3435 |
| CB-15 | 464.6 | G-site | −7.2967 |
| CB-16 | 492.6 | G-site | −6.9912 |
| CB-17 | 371.3 | G-site | −6.4278 |
| CB-18 | 416.5 | G-site | −6.0457 |
| CB-19 | 429.9 | G-site | −6.7879 |
| CB-20 | 378.4 | G-site | −7.4946 |
| CB-21 | 473.5 | H site | −15.9 |
| CB-22 | 461.5 | H site | −16.57 |
| CB-23 | 433.4 | H site | −16.68 |
| CB-24 | 474.5 | H site | −18.55 |
| CB-25 | 462.5 | H site | −16.74 |
| CB-26 | 403.5 | H site | −15.94 |
| CB-27 | 444.5 | H site | −16.13 |
| CB-28 | 442.5 | H site | −16.15 |
| CB-29 | 417.6 | H site | −16.57 |
| CB-30 | 393.4 | H site | −16.28 |
| CB-31 | 447.6 | H site | −17.38 |
| CB-32 | 439.5 | H site | −16.25 |
| CB-33 | 506.6 | H site | −13.65 |
| CB-34 | 419.4 | H site | −15.84 |
| CB-35 | 472.5 | H site | −16.41 |
| CB-36 | 382.5 | H site | −13.45 |
| CB-37 | 340.5 | H site | −14.61 |
| CB-38 | 428.4 | H site | −16.53 |
| CB-39 | 314.4 | H site | −12.18 |
| CB-40 | 320.4 | H site | −12.94 |

### Example 2—In Vitro Antimalarial Activity in P. berghei Parasites

Materials and Methods. The P. berghei ANKA 507cl1 expressing green fluorescent protein (GFP) and P. berghei GFP-Lucamai (1037cl1) expressing GFP and the firefly luciferase (luc) gene were used. The in vitro drug luminescence (ITDL) assay was used to assess antimalarial activity and the effective concentration (EC₅₀) of the identified compounds using the P. berghei GFP-Lucamai (1037cl1) parasite line. Chloroquine diphosphate salt (CQ) from Sigma Aldrich® was used as a control at 100 nM concentration. The ChemBridge Hit2Lead library compounds were purchased in powder form and dissolved in 100% DMSO to obtain a 10 mM stock solution, aliquot and stored at −20° C. Compounds dilutions were prepared in complete culture medium (RPMI1640 medium supplemented with 20% FBS from Gibco® heat-inactivated and Neomycin stock solution of 10,000 IU/mL from Sigma-Aldrich®) within 24 hours prior to initiation of the experiment and stored at 4° C. The initial screening was carried out using 10 μM of each compound in triplicate. Compounds that inhibit >50% of parasite growth at 10 μM were further used in a dose-response analysis and the EC₅₀ was determined using at least eight compound concentrations. Data analysis and the EC₅₀ calculation were done using Microsoft Excel and GraphPad Prism 6 software, respectively. Dose-response curves of at least four independent experiments in triplicate each are reported.

In vitro susceptibility of the compound CB-27 was done in the P. falciparum multidrug-resistant Dd2 clone B2. Briefly, most ring-stage parasites were incubated at 0.2% starting parasitemia and 1% hematocrit with a range of compound CB-27 concentrations at 37° C. for 72 hours in 96-well plates. After 72 hours, parasite growth was assessed using flow cytometry on an Accuri C6 cytometer with parasites stained with SYBR green I and MitoTracker Deep Red. Compound CB-27 was tested in six independent experiments with technical replicates. The percentage of parasite growth was curve fitted against log-transformed drug concentrations and the EC₅₀ was calculated using GraphPad Prism 6 software.

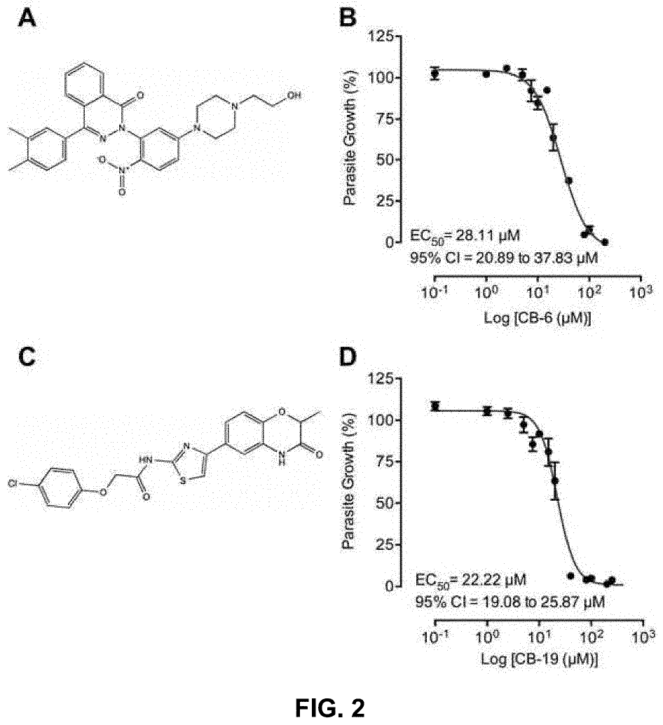

Results. Biological evaluation of 40 small compounds revealed that three (CB-6, CB-19, and CB-27) of them inhibited >50% of parasite growth at the cutoff concentration of 10 μM (FIGS. 1B and 1C). Dose-response curves from CB-6 and CB-19 showed EC₅₀'s of 28.11 μM and 22.22 μM, respectively (FIG. 2). Interestingly, CB-27 inhibited P. berghei and P. falciparum multidrug-resistant Dd2 clone B2 parasites growth at 0.505 μM and 0.958 μM (FIG. 3B).

### Example 3—Plasmodium berghei Glutathione S-Transferase Inhibition

Materials and methods. GST inhibition was determined in crude protein extracts from P. berghei ANKA 507cl1 blood stages according to the following method. Parasite extracts were prepared and the pellets containing proteins were resuspended in buffer (3.5 mM MgCl₂, 110 mM KCl, 40 mM NaCl, 20 mM HEPES, 6 mM EDTA, pH 7.4) with protease inhibitors (0.01 mg of leupeptin A, 0.001 mg of pepstatin A, 0.35 mg of PMSF). The parasite pellets were lysed by three freeze/thaw cycles (liquid nitrogen and 37° C. water bath) and protein content was determined using Bio-Rad DC Protein Assay. PbGST inhibition by compound CB-27 was determined by adding variable concentrations of the compound (1, 10, and 50 μM) with 0.65 mg/mL of P. berghei protein extracts in 200 μL of total volume containing 1 mM of 1-chloro-2,4-dinitrobenzene (CDNB) from Sigma-Aldrich® and 100 mM potassium phosphate buffer (pH 6.5) at 25° C. Compound CB-27 dilutions were prepared in 0.5% DMSO as final concentration. The reaction was initiated by the addition of 1 mM GSH and the formation of S-(2,4-dinitrophenyl)glutathione was monitored spectrophotometrically at 340 nm (ε340 nm=0.0096 μM⁻¹cm⁻¹) using the SpectraMax M3 Microplate Reader (Molecular Devices). Inhibition of PbGST by compound CB-27 was done in four independent experiments (one replicate each).

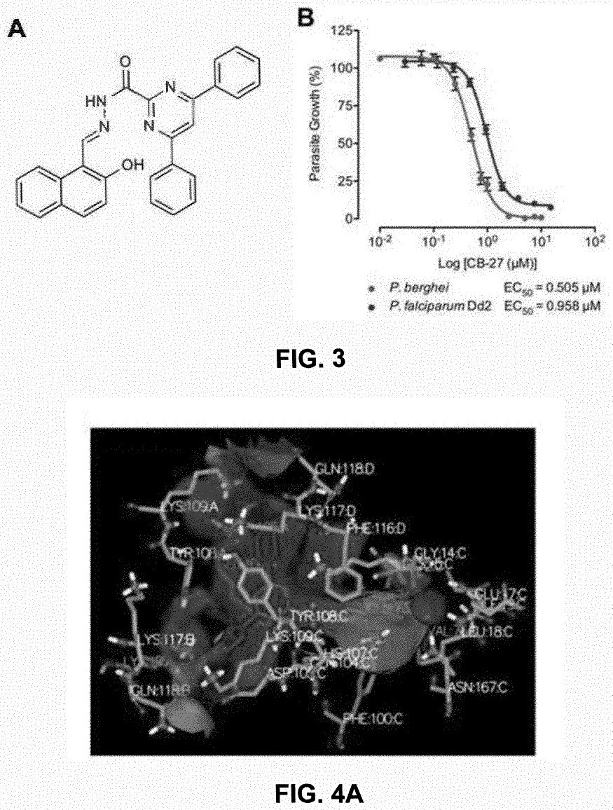

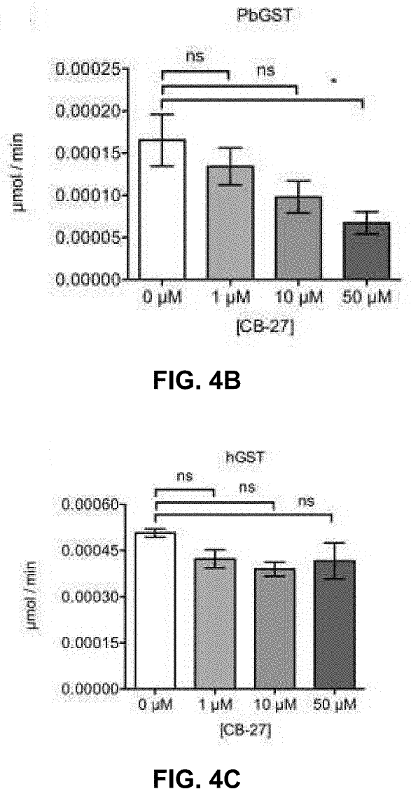

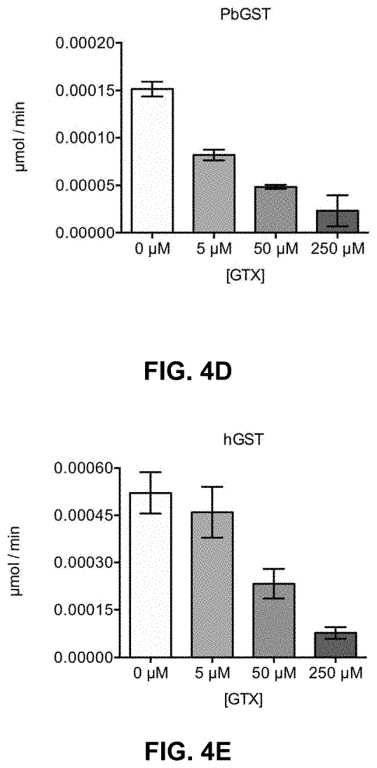

Results. CB-27 was modeled into the PbGST H-site and docking analysis revealed the predicted binding mode and amino acids interactions (FIG. 4A). CB-27 is represented as sticks and the amino acids interacting are shown in three letter code. The inhibitory activity of CB-27 was determined using an in vitro GST inhibition assay in a crude P. berghei ANKA 507cl1 protein extract from blood stages. As shown in FIG. 4B, CB-27 displayed a concentration-dependent inhibition of PbGST. As shown in FIG. 4C, CB-27 does not inhibit human GST. As a positive control, PbGST and human GST inhibition were determined in the presence of a specific GST inhibitor, S-hexylglutathione, as shown in FIGS. 4D and 4E, respectively.

### Example 4—Shape Similarity Screening

Materials and methods. A shape similarity screening was done using the ROCS tool (version 3.2.2.2) from the OpenEye Scientific software package and the ChemBridge Hit2Lead library. Multi-conformer files were generated by OMEGA and saved in oeb.gz format. The multi-conformational files were used to carry out a Rapid Overlay of Chemical Structures (ROCS) similarity search. ROCS use a smooth Gaussian function to identify ligands using a shape-based superimposition methods to find similar but nonintuitive compounds. For the analysis, ROCS use the heavy atoms ignoring the hydrogens. The output files of the shape similarity screening reports rigorous Tanimoto and Tversky measure between shapes and were ranked according to their combo score based on 3D shape and chemical properties. The ligands obtained by shape similarity screening were also docked to the PbGST H binding site to confirm interaction into the predicted H binding site.

Results. A shape similarity screening was done to identify other chemical scaffolds similar to CB-27 which could inhibit PbGST resulting in antimalarial activity. Results from the shape similarity screening were ranked according to their ROCS combo score as provided in Table 2.

TABLE 2

| ID | g/mol | ROCS Combo Score |
|---|---|---|
| CB-41 | 424.5 | 1.9300 |
| CB-42 | 408.5 | 1.6750 |
| CB-43 | 396.4 | 1.6110 |
| CB-44 | 408.5 | 1.5370 |
| CB-45 | 379.4 | 1.5640 |
| CB-46 | 413.4 | 1.4820 |
| CB-47 | 432.9 | 1.5250 |
| CB-48 | 402.5 | 1.3480 |
| CB-49 | 411.5 | 1.3340 |
| CB-50 | 490.3 | 1.3250 |
| CB-51 | 417.6 | 1.3700 |
| CB-52 | 434.5 | 1.3170 |
| CB-53 | 367.4 | 1.3370 |
| CB-54 | 453.9 | 1.3200 |
| CB-55 | 423.5 | 1.2690 |
| CB-56 | 381.4 | 1.2990 |
| CB-57 | 447.6 | 1.3180 |
| CB-58 | 397.4 | 1.2920 |
| CB-59 | 440.9 | 1.3360 |
| CB-60 | 396.5 | 1.3360 |
| CB-61 | 380.3 | 1.3060 |
| CB-62 | 504.6 | 1.3480 |
| CB-63 | 353.4 | 1.2990 |
| CB-64 | 412.9 | 1.2900 |

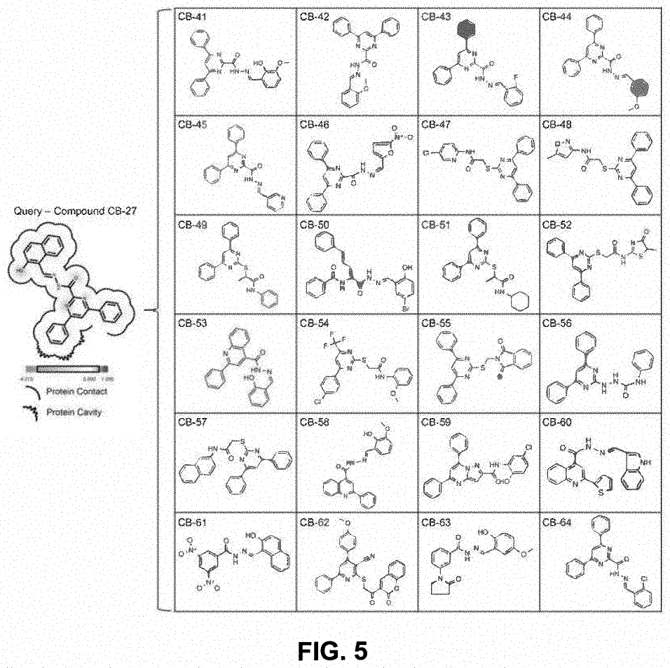

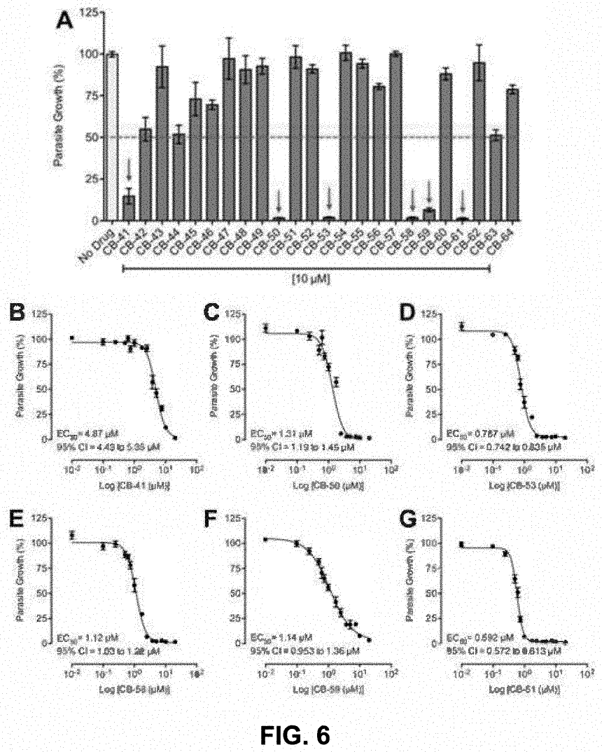

A total of 24 compounds were chosen from the set of docking-generated decoys with similar shape, chemical constraints, and electrostatics parameters (FIG. 5). Those 24 compounds were prioritized for biological evaluation for their effect on parasite survival and six compounds (CB-41, CB-50, CB-53, CB-58, CB-59, and CB-61) showed inhibition >50% of parasite growth at 10 μM (FIG. 6A). Dose-response curves revealed that six compounds (CB-41, CB-50, CB-53, CB-58, CB-59, and CB-61) exhibited antiplasmodial activity against P. berghei at low micromolar concentrations (EC₅₀ of ˜0.6-˜4.9 μM) (FIG. 6B-6G, Table 3).

TABLE 3

| Compound | EC₅₀ | 95% CI |
|---|---|---|
| CB-27 | 0.5 | 0.46 to 0.5 |
| CB-41 | 4.9 | 4.4 to 5.3 |
| CB-50 | 1.3 | 1.2 to 1.5 |
| CB-53 | 0.8 | 0.7 to 0.8 |
| CB-58 | 1.1 | 1.0 to 1.2 |
| CB-59 | 1.1 | 0.9 to 1.4 |
| CB-61 | 0.6 | 0.5 to 0.6 |

### Example 5—Red Blood Cell Lysis Assay

Materials and methods. Random-bred Swiss albino CD-1 female mice (Charles River Laboratories, Wilmington, Mass., USA) from 6-8 weeks old were used for the study. All mice procedures were approved by the Institutional Animal Care and Use Committee under the protocol number 2480108 at the AAALAC accredited University of Puerto Rico-Medical Sciences Campus Animal Resources Center. The mice work was done in strict accordance with the “Guide for the Care and Use of Laboratory Animals” (National-Research-Council, Current Edition) and regulations of the PHS Policy on Humane Care and Use of Laboratory Animals. Mice were acclimated for 1 week before initiation of experiments.

Compounds were analyzed at 10 serial dilutions using fresh mouse erythrocytes at 1% hematocrit in Dulbecco's PBS (Gibco®) in V-bottom microplates (Corning® 96 well TC-treated microplate). The plates were incubated for 24 hours at 37° C. followed by centrifugation at 2,000 rpm for 5 min, and 50 μl of supernatant was transferred to a fresh flat-bottom microplate (BD Falcon®). The amount of hemoglobin release in the supernatant was determined using the QuantiChrom™ Hemoglobin Assay Kit (DIHB-250; BioAssay Systems) by measuring the absorbance at 400 nm following the manufacturer's instructions. Saponin at 100 μg/ml was used as a positive control for 100% cell lysis, blood (1% hematocrit) with Dulbecco's Phosphate-Buffered Saline (DPBS) as a negative control for no cell lysis, and DPBS as a blank. Compounds were tested in three independent experiments in triplicate each.

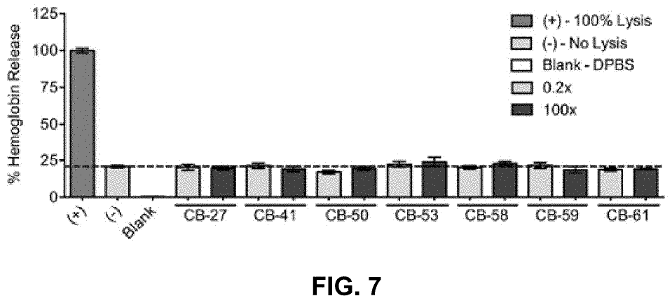

Results. As discussed above, the seven identified compounds were shown to inhibit P. berghei intra-erythrocytic growth. Since Plasmodium parasites live inside red blood cells for the majority of their life cycle, it is important to assess whether the compounds cause lysis to the cells. The red blood cell lysis potential was evaluated in the identified antimalarial compounds (CB-27, CB-41, CB-50, CB-53, CB-58, CB-59 and CB-61) at ten serial dilutions from 0.2-100× fold above their EC₅₀'s (FIG. 7). None of the identified compounds (CB-27, CB-41, CB-50, CB-53, CB-58, CB-59 and CB-61) cause hemolysis at their EC₅₀'s values. Further, the results showed that the compounds did not hemolyze erythrocytes even at concentrations higher (100× fold) than their EC₅₀'s, demonstrating that the antiplasmodial effects of the compounds are not due to toxicity against erythrocytes.

### Example 6—Predicted Pharmacokinetic and Toxicity Properties

Materials and methods. The pharmacokinetic and toxicity properties were predicted using the pkCSM server (http://biosig.unimelb.edu.au/pkcsm/prediction) that used graph-based signatures to develop predictive regression and classification models. The pkCSM used the SMILE string to predict absorption, distribution, metabolism, excretion, and toxicological parameters (ADMET). Analysis and interpretation of pharmacokinetic and toxicity properties results was performed as recommended by Pires and colleagues (http://biosig.unimelb.edu.au/pkcsm/theory).

Results. The predicted pharmacokinetics and toxicity properties of the identified compounds (CB-27, CB-41, CB-50, CB-53, CB-58, CB-59 and CB-61) were assessed and compared to chloroquine (CQ). The results are summarized in Table 3.

TABLE 3

| Parameters | Predictors | CQ | CB-27 | CB-41 | CB-50 | CB-53 | CB-58 | CB-59 | CB-61 | Unit |
|---|---|---|---|---|---|---|---|---|---|---|
| Absorption | Water | −4.249 | −4.627 | −4.913 | −4.229 | −4.891 | −4.729 | −3.554 | −4.675 | log mol/L |
| | solubility | | | | | | | | | |
| | Caco2 | 1.624 | 0.875 | 0.533 | 0.605 | 0.538 | 0.546 | 0.985 | −0.22 | log Papp |
| | Intestinal | 89.95 | 94.826 | 100 | 88.904 | 93.649 | 94.756 | 94.135 | 90.3 | % |
| | abs | | | | | | | | | Absorbed |
| | Skin perm | −2.679 | −2.735 | −2.734 | −2.737 | −2.737 | −2.739 | −2.735 | −2.739 | log Kp |
| | Pgp subs | Yes | Yes | Yes | Yes | Yes | Yes | Yes | Yes | Yes/No |
| | Pgp I inh | No | Yes | Yes | Yes | Yes | Yes | Yes | Yes | Yes/No |
| | Pgp II inh | No | Yes | Yes | Yes | Y'es | Yes | Yes | Yes | Yes/No |
| Distribution | VDss | 1.332 | −0.279 | −0.428 | 0.127 | −0.427 | −0.417 | −0.065 | −0.48 | log L/kg |
| | Fraction | 0.191 | 0.185 | 0.02 | 0 | 0 | 0 | 0.237 | 0 | Fu |
| | unbound | | | | | | | | | |
| | BBB perm | 0.349 | 0.143 | −0.17 | −0.702 | −0.138 | −0.35 | −0.333 | −0.659 | log BB |
| | CNS perm | −2.191 | −1.719 | −2.153 | −2.08 | −1.775 | −1.946 | −1.569 | −2.381 | log PS |
| Metabolism | CYP2D6 | Yes | No | No | No | No | No | No | No | Yes/No |
| | subs | | | | | | | | | |
| | CYP3A4 | Yes | Yes | Yes | Yes | Yes | Yes | Yes | Yes | Yes/No |
| | subs | | | | | | | | | |
| | CYP1A2 | No | Yes | No | No | Yes | Yes | Yes | Yes | Yes/No |
| | inh | | | | | | | | | |
| | CYP2C19 | No | Yes | Yes | Yes | Yes | Yes | Yes | Yes | Yes/No |
| | inh | | | | | | | | | |
| | CYP2C9 | No | Yes | Yes | Yes | Yes | Yes | Yes | No | Yes/No |
| | inh | | | | | | | | | |
| | CYP2D6 | Yes | No | No | No | No | No | No | No | Yes/No |
| | inh | | | | | | | | | |
| | CYP3A4 | No | Yes | Yes | Yes | Yes | Yes | Yes | Yes | Yes/No |
| | inh | | | | | | | | | |
| Excretion | Total | 1.092 | 0.84 | 0.777 | −0.257 | 0.646 | 0.59 | 0.319 | 0.297 | log |
| | clearance | | | | | | | | | ml/min/kg |
| | Renal | Yes | No | No | No | No | No | No | No | Yes/No |
| | OCT2 subs | | | | | | | | | |
| Toxicity | AMES | Yes | Yes | Yes | No | No | Yes | No | Yes | Yes/No |
| | Max tol | −0.167 | 0.664 | 0.334 | −0.105 | 0.147 | 0.205 | 0.747 | −0.122 | log |
| | dose | | | | | | | | | mg/kg/day |
| | hERG I inh | No | No | No | No | No | No | No | No | Yes/No |
| | hERG II inh | Yes | Yes | Yes | Yes | Yes | Yes | Yes | Yes | Yes/No |
| | Oral rat | 2.85 | 3.781 | 2.909 | 2.239 | 2.474 | 2.456 | 3.186 | 2.6 | mol/kg |
| | LD50 | | | | | | | | | |
| | Oral rat | 1.026 | 0.424 | 1.884 | 2.84 | 2.363 | 2.482 | 1.559 | 2.998 | log |
| | LOAEL | | | | | | | | | mg/kg_b |
| | | | | | | | | | | w/day |
| | Hepato- | Yes | Yes | Yes | Yes | Y'es | Yes | Yes | Yes | Yes/No |
| | toxicity | | | | | | | | | |
| | Skin sens | No | No | No | No | No | No | No | No | Yes/No |
| | T. | 1.558 | 0.285 | 0.291 | 0.388 | 0.326 | 0.31 | 0.285 | 0.326 | Numeric |
| | pyriformis | | | | | | | | | (log μg/L) |
| | Minnow | 0.747 | −1.52 | −1.59 | 0.34 | −0.944 | −1.032 | −1.435 | 0.304 | Numeric |
| | | | | | | | | | | (log mM) |

Parameters associated with absorption such as water solubility, membrane permeability in colon cancer cell line (Caco2), intestinal absorption, skin permeability levels, and P-glycoprotein substrate or inhibitor (Pgp subs, Pgp I/II inh) were calculated, and the identified seven compounds (CB-27, CB-41, CB-50, CB-53, CB-58, CB-59 and CB-61) were predicted to be water-soluble with values within −3.554 to −4.913 log mol/L, similar to the predicted value of CQ (−4.249 log mol/L). Caco2 permeability is considered high when Papp coefficient is >8×10⁻⁶, and the predicted value is >0.90. Therefore, CB-27 and CB-59 were predicted to have high Caco2 permeability. Compounds displaying absorbance of less than 30% are considered to have reduced intestinal absorption. The seven compounds were predicted to have high absorption with estimated values that ranged from 88.9 to 100%, similar to CQ (89.95%). Skin permeability is vital for transdermal drug delivery, and a log Kp>−2.5 is considered to have relatively low skin permeability. Like CQ, all seven compounds were predicted to be skin permeable. The seven compounds and CQ were predicted to be both P-glycoprotein (PgP) substrates and PgP I/II inhibitors.

Four predictors of drug distribution, including volume of distribution (VDss), fraction unbound, blood-brain barrier (BBB) permeability, and Central Nervous System (CNS) permeability were assessed. The VDss is the theoretical volume that a drug needs to be uniformly distributed to produce the same plasma concentration. The VDss is estimated low when log VDss<−0.15 and high when log VDss>0.45. Except for CB-50 and CB-59, the other compounds had an estimated low VDss. CB-27 and CB-59 had predicted values for unbound fraction of 0.185 and 0.237, respectively; which are similar to CQ (0.191). BBB permeability, and CNS permeability can estimate drug distribution into the brain. Compounds with log BB>0.3 are suggested to readily cross the BBB while compounds with log BB<−1 cross poorly. Like CQ, all seven identified compounds were predicted to cross the BBB with values that ranged from −0.702 to 0.143 log BB. The blood-brain permeability-surface area product (log PS) measures CNS permeability in which compounds with a log PS>−2 are suggested to penetrate the CNS while those with log PS<−3 are unable to penetrate the CNS. All seven identified compounds were predicted to penetrate the CNS.

Drug metabolism was predicted based on the CYP models for substrate or inhibition. Results show that the seven identified compounds are predicted substrates for CYP3A4 and were predicted to inhibit the isoenzymes CYP2C19, and CYP3A4. Drug excretion was measured using two predictors, the renal OCT2 substrate predictor that describes the potential of a drug to be secreted by the kidney, and total clearance that combines hepatic clearance and renal clearance. The predicted renal OCT2 data suggest that the seven identified compounds are non-substrates of the OCT2 pathway. Differences in predicted total clearance can be observed between the identified compounds with estimated values that range from −0.257 to 0.84 log ml/min/kg. Total clearance predictions show all seven identified compounds with a lower total clearance than CQ and, that of the seven identified compounds, CB-27 has the predicted highest total clearance followed by CB-41, CB-53, and CB-58.

The AMES test (carcinogenicity), hERG inhibition (cardiotoxicity), hepatotoxicity, and skin sensitization were used to predict the toxicity of the seven identified compounds. According to AMES toxicity prediction, CB-50, CB-53, and CB-59 are not mutagenic. However, CQ and CB-27, CB-41, CB-58, and CB-61 have a predicted positive AMES test. The predictions suggest that all seven identified compounds and CQ are hERG II inhibitors. The predictions suggest that all seven identified compounds and CQ may have hepatotoxic potential but do not cause skin sensitization.

In summary, the seven identified compounds inhibit P. berghei intra-erythrocytic growth, and none induce hemolysis. The hemolytic activity analysis revealed that these identified compounds are not toxic to erythrocytes. All seven identified compounds are predicted to fulfill the absorption requirements. The predicted differences between the identified compounds and CQ in P-glycoprotein modulation, and CYP2D6 and CYP3A metabolism suggests that the identified compounds present favorable and less metabolism-based drug interactions. Drug distribution predictors, BBB permeability and CNS permeability, suggest that the identified compounds can cross the BBB comparable to CQ and supports its use to treat cerebral malaria. According to drug excretion predictions, CB-27 has the highest total clearance, and all identified compounds are not renal OCT2 substrates. The predictions suggest no differences between CQ and the identified compounds in terms of inhibition of hERG I/II, and hepatotoxicity. These identified compounds have drug-like properties with acceptable pharmacokinetic profiles for oral route due to their predictions of high intestinal absorption, metabolism in the liver, drug distribution into the brain, and low excretion. Results from pharmacokinetic and toxicity predictions suggest that ADMET profiles are similar to CQ.

1. A method of inhibiting growth of a Plasmodium species comprising:
contacting a Plasmodium species with a compound selected from the group consisting of formulae I, II, and III: 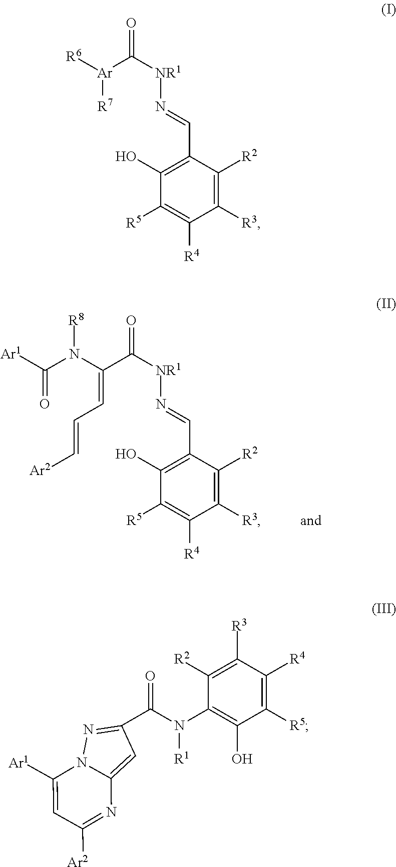
wherein R¹ is H or C₁₋₃ alkyl;
R², R³, R⁴, and R⁵ are independently H, C₁₋₃ alkyl, C₁₋₃ alkoxy, F, Cl, Br, and I, or
R² and R³, R³ and R⁴, or R⁴ and R⁵, taken together with the carbon atoms to which they are attached, form a 5- or 6-membered aryl or heteroaryl ring;
Ar is a 5- to 10-membered aryl or heteroaryl ring;
R⁶ is H, NO₂, or a 5- or 6-membered aryl or heteroaryl ring;
R⁷ is NO₂ or a 5- or 6-membered aryl or heteroaryl ring;
R⁸ is H or C₁₋₃ alkyl; and
Ar¹ and Ar² are independently NO₂ or a 5- or 6-membered aryl or heteroaryl ring.

2. The method of claim 1, wherein R¹ is H.

3. The method of claim 1, wherein R² and R³, together with the carbon atoms to which they are attached, form a benzene ring.

4. The method of claim 3, wherein R⁴ and R⁵ are H.

5. The method of claim 1, wherein at least one of R², R³, R⁴, and R⁵ is methoxy.

6. The method of claim 5, wherein the others of R², R³, R⁴, and R⁵ are H.

7. The method of claim 1, wherein R⁵ is methoxy and R², R³, and R⁴ are H.

8. The method of claim 1, wherein R², R³, R⁴, and R⁵ are H.

9. The method of claim 1, wherein Ar is phenyl, pyridinyl, pyridazinyl, pyrimidinyl, pyrazinyl, triazinyl, pyrrolyl, imidazolyl, pyrazolyl, triazolyl, quinolinyl, or isoquinolinyl.

10. The method of claim 1, wherein Ar is

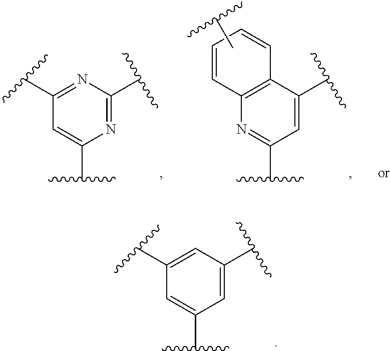

11. The method of claim 9, wherein R⁶ and R⁷ are both NO₂.

12. The method of claim 1, wherein R⁶ and R⁷ are both phenyl.

13. The method of claim 1, wherein R⁶ is H and R⁷ is phenyl.

14. The method of claim 1, wherein Ar¹ and Ar² are both phenyl.

15. The method of claim 14, wherein R⁸ is H.

16. The method of claim 15, wherein one of R², R³, R⁴, and R⁵ is Br and the others of one of R², R³, R⁴, and R⁵ are H.

17. The method of claim 16, wherein R³ is Br and R², R⁴, and R⁵ are H.

18. The method of claim 15, wherein one of R², R³, R⁴, and R⁵ is Cl and the others of one of R², R³, R⁴, and R⁵ are H.

19. The method of claim 18, wherein R³ is Cl and R², R⁴, and R⁵ are H.

20. The method of claim 1, wherein the compound is selected from the group consisting of:

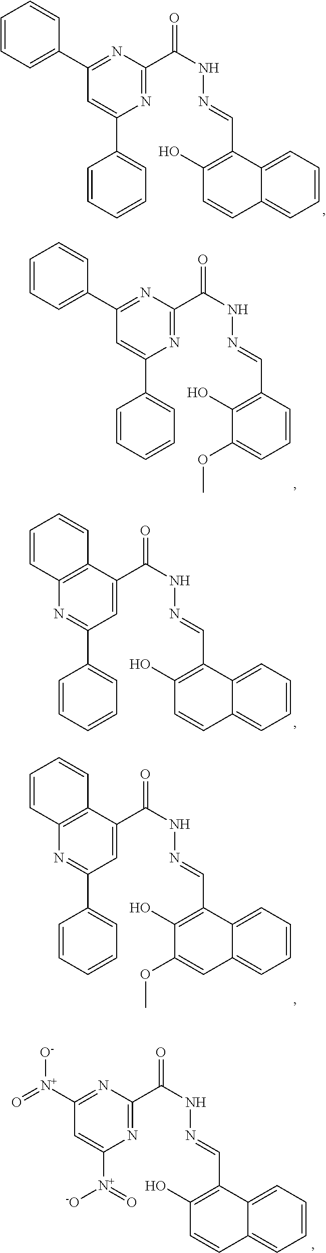

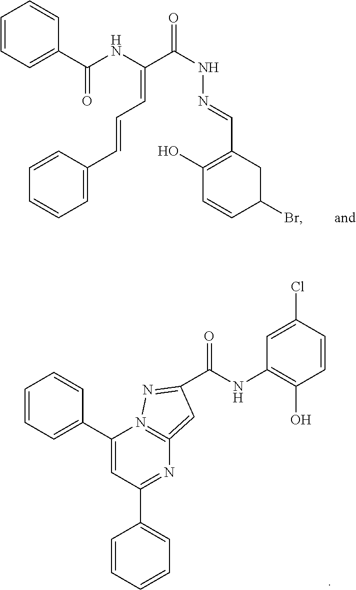

21. The method of claim 1, wherein the Plasmodium species is Plasmodium falciparum, Plasmodium vivax, Plasmodium malariae, Plasmodium ovale, Plasmodium knowlesi, or Plasmodium berghei.

22. The method of claim 1, wherein the Plasmodium species is a multidrug-resistant Plasmodium strain.

23. A method for treating malaria comprising:
administering a compound to a human or animal patient in need thereof, wherein the compound is selected from the group consisting of formulae I, II, and III: 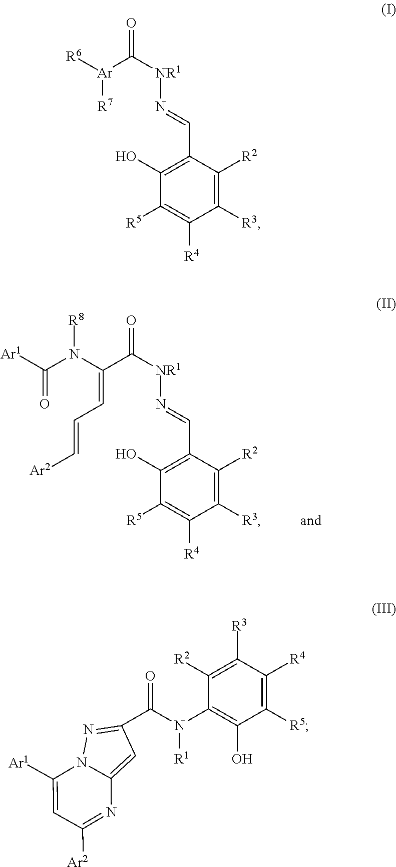
wherein R¹ is H or C₁₋₃ alkyl;
R², R³, R⁴, and R⁵ are independently H, C₁₋₃ alkyl, C₁₋₃ alkoxy, F, Cl, Br, and I, or R² and R³, R³ and R⁴, or R⁴ and R⁵, taken together with the carbon atoms to which they are attached, form a 5- or 6-membered aryl or heteroaryl ring;
Ar is a 5- to 10-membered aryl or heteroaryl ring;
R⁶ is H, NO₂, or a 5- or 6-membered aryl or heteroaryl ring;
R⁷ is NO₂ or a 5- or 6-membered aryl or heteroaryl ring;
R⁸ is H or C₁₋₃ alkyl; and
Ar¹ and Ar² are independently NO₂ or a 5- or 6-membered aryl or heteroaryl ring.

24. The method of claim 23, further comprising co-administering a compound selected from the group consisting of chloroquine, atovaquone-proguanil, artemether-lumefantrine, mefloquine, quinine, quinidine, doxycycline, clindamycin, artesunate, and combinations thereof.

25. A method for inhibiting a glutathione S-transferase (GST) comprising:
contacting a GST with a compound of selected from the group consisting of formulae I, II, and III: 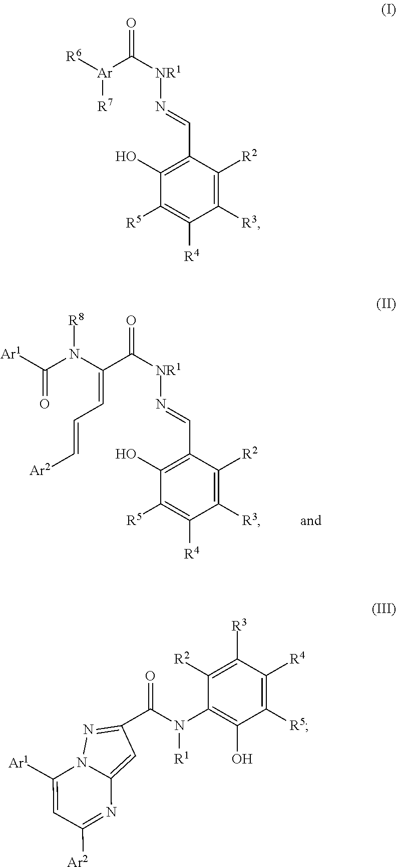
wherein R¹ is H or C₁₋₃ alkyl;
R², R³, R⁴, and R⁵ are independently H, C₁₋₃ alkyl, C₁₋₃ alkoxy, F, Cl, Br, and I, or
R² and R³, R³ and R⁴, or R⁴ and R⁵, taken together with the carbon atoms to which they are attached, form a 5- or 6-membered aryl or heteroaryl ring;
Ar is a 5- to 10-membered aryl or heteroaryl ring;
R⁶ is H, NO₂, or a 5- or 6-membered aryl or heteroaryl ring;
R⁷ is NO₂ or a 5- or 6-membered aryl or heteroaryl ring;
R⁸ is H or C₁₋₃ alkyl; and
Ar¹ and Ar² are independently NO₂ or a 5- or 6-membered aryl or heteroaryl ring.

26. The method of claim 25, wherein the GST is from a Plasmodium species.

27. A compound selected from the group consisting of formulae I, II, and III: 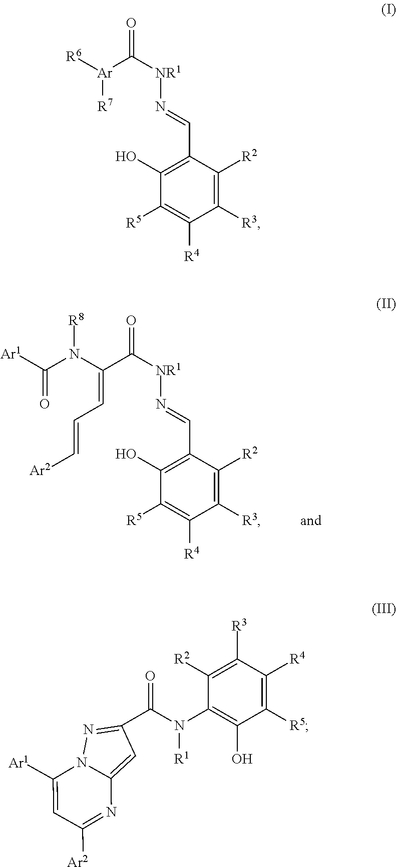
wherein R¹ is H or C₁₋₃ alkyl;
R², R³, R⁴, and R⁵ are independently H, C₁₋₃ alkyl, C₁₋₃ alkoxy, F, Cl, Br, and I, or
R² and R³, R³ and R⁴, or R⁴ and R⁵, taken together with the carbon atoms to which they are attached, form a 5- or 6-membered aryl or heteroaryl ring;
Ar is a 5- to 10-membered aryl or heteroaryl ring;
R⁶ is H, NO₂, or a 5- or 6-membered aryl or heteroaryl ring;
R⁷ is NO₂ or a 5- or 6-membered aryl or heteroaryl ring;
R⁸ is H or C₁₋₃ alkyl; and
Ar¹ and Ar² are independently NO₂ or a 5- or 6-membered aryl or heteroaryl ring;
with the proviso that the compound is not 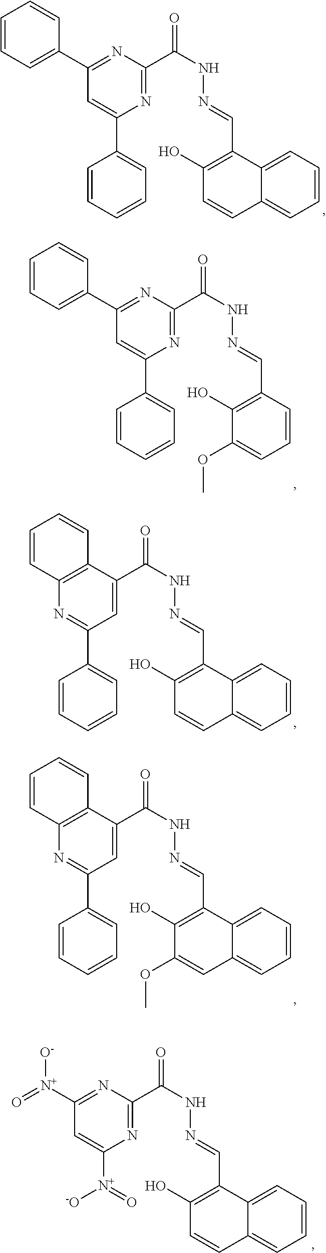 [chemical structure]
